(19) United States
(12) Patent Application Publication
(10) Pub. No.: US 2015/0272750 A1
(43) Pub. Date: Oct. 1, 2015

(54) Expandable Device

(71) Applicant: ICON MEDICAL CORP., Atlanta, GA (US)

(72) Inventor: Noah Roth, Atlanta, GA (US)

(21) Appl. No.: 14/433,122

(22) PCT Filed: Oct. 1, 2013
(86) PCT No.: PCT/US13/62804

§ 371 Date: Apr. 2, 2015

Related U.S. Application Data
(60) Provisional application No. 61/708,878, filed on Oct. 2, 2012.

(51) Int. Cl. A61F 2/82

(52) U.S. Cl. CPC A61F 2/82; A61F 2230/0069

(57) ABSTRACT

A medical device that is formed of a backbone portion and a plurality of ribs. The ends of one or more of the ribs are not connected together when the medical device is in the expanded position.

The present inventions claims priority on U.S. Provisional Patent Application Ser. No. 61/708,878 filed Oct. 2, 2012, which is incorporated herein by reference.

The invention relates generally to medical devices, and more particularly to an implant for use within a body, and even more particularly to an expandable graft which is particularly useful for repairing various types of body passageways.

### BACKGROUND OF THE INVENTION

Medical treatment of various illnesses or diseases commonly includes the use of one or more medical devices. These devices can be implanted in various areas of the mammalian anatomy. One purpose of a stent is to open a blocked or partially blocked body passageway. When used in a blood vessel, the stent opens the occluded vessel to achieve improved blood flow, which is necessary for the anatomical function of an organ. The procedure of opening a blocked or partially blocked body passageway commonly includes the use of one or more stents in combination with other medical devices such as, but not limited to, an introducer sheath, a guiding catheter, a guide wire, an angioplasty balloon, etc.

Old age, dietary habits and primary genetics can also lead to a common disease, atherosclerosis. Atherosclerotic plaques and blockages consist of lipids, fibroblasts and fibrin that proliferate and cause obstruction of a vessel. As the obstruction grows, the blood flow diminishes and reaches a level that is insufficient to meet the biological needs of one or more organs. The end result is defined as ischemia.

One purpose of an expandable balloon and a stent is to open a blocked or partially blocked body passageway. When used in a blood vessel, the balloon and/or stent is opens the occluded vessel to achieve improved blood flow, which is necessary for the anatomical function of an organ. The procedure of opening a blocked or partially blocked body passageway commonly includes the use of a balloon or stents in combination with other medical devices such as, but not limited to, an introducer sheath, a guiding catheter, a guide wire, etc.

Alternatively, congenital defects often result in pre-existing obstructions in the passageway of the body. Specifically, a defect in the formation of collagen rings in the trachea result in a floppy trachea that collapses during respiration. One use of a stent is to hold open the trachea during respiration.

### SUMMARY OF THE INVENTION

The present invention is directed to medical devices and more particularly to expandable devices (e.g., stents, etc.) that are insertable into body passageways (e.g., blood vessels, trachea, etc.). The medical device is designed to be expanded when inserted into a treatment area. The expansion of the medical device is designed to increase the size of an opening, unblock an opening, and/or provide structural support in the body passageway. The medical device is novel over past expandable medical devices in that the body of the medical device along the longitudinal length of the medical device is not continuous about the complete circumference of the body of the medical device.

In one non-limiting embodiment of the invention, the medical device includes a body having one or more backbone portions and one or more rib portions. The one or more backbone portions are designed to connect together the one or more rib portions. The backbone portion can have a generally rectangular shape; however, this is not required. The thickness, width and shape of the backbone portion are non-limiting. The backbone portion can be a solid piece of material, can have one or more openings, be a mesh design, etc. The backbone portion runs along the longitudinal length of the body of the medical device. A plurality of rib portions are connected to or formed on the backbone portion. The rib portions are generally spaced apart at equal distances; however, this is not required. The number of rib portions on the backbone portion is non-limiting. The rib portions can be the same shape and size; however, this is not required. The length, width and shape of the rib portions are non-limiting. Prior to the medical device being inserted into the body passageway, the rib portions and/or the backbone portion can be bent so that the ends of the rib portions overlap one another or are positioned adjacent to one another; for instance, the rib portions and the backbone portion can be bent to form a cross-sectional C-shape. As can be appreciated, other cross-section shapes can be formed. Generally, the ends are not connected together; however, the first and second side edges can be designed to be releasably connected together. A balloon catheter can be used to expand the medical device in the body passageway; however, this is not required. The materials used to form the rib portions and/or the backbone portion is non-limiting (e.g., metal, polymer, etc.).

In still another non-limiting embodiment of the invention, the medical device can include a body having first and second side edges and front and rear edges. The first and second side edges can run along the longitudinal length of the body and terminate at the front and rear edges of the body. The body of the medical device can be bent or formed to cause the first and second side edges to overlap one another or be positioned adjacent to one another; however, the first and second side edges are designed to be movable with respect to one another when the medical device is expanded in the body passageway. Generally, the first and second side edges are not connected together; however, the first and second side edges can be designed to be releasably connected together. The body of the medical device can be a solid piece of material, can have one or more openings, be a mesh design, etc. The materials used to form the body portion are non-limiting (e.g., metal, polymer, etc.).

In one non-limiting aspect of the invention, there is provided an expandable medical device that is designed to be inserted into a body passageway and to be expandable in the body passageway once the medical device is inserted at the desired treatment area in the body passageway. The medical device includes a backbone portion and a plurality of ribs. The backbone portion extends a majority the longitudinal length of the medical device, typically at least about 60% the longitudinal length of the medical device the longitudinal length of the medical device, more typically at least about 75% the longitudinal length of the medical device, still more typically at least about 90% the longitudinal length of the medical device, and yet more typically about 100% the longitudinal length of the medical device. The width of the backbone portion is non-limiting. Generally, the width of the backbone portion is such that is consists of no more than about 75% of the outer circumference of the medical device in the fully expanded position, typically about 5-70% of the outer circumference of the medical device in the fully expanded position, and more typically about 10-65% of the outer circumference of the medical device in the fully expanded position. In one non-limiting arrangement, the width of the backbone portion is such that is consists of less than 50% of the outer circumference of the medical device in the fully expanded position. The backbone portion can be formed of a solid piece of material, be formed of a mesh material, be formed of a plurality of pieces of material, etc. The shape of the backbone portion is non-limiting (e.g., rectangular, square polygonal, oval, circular, etc.). The thickness of the backbone portion is non-limiting.

In one non-limiting arrangement, the backbone portion has a generally uniform thickness. In another non-limiting arrangement, the backbone portion has a thickness that is the same or is greater than the thickness of one or more of the ribs that are connected to the backbone portion. The plurality of ribs that are connected to the backbone portion have a non-limiting size, shape, length, width and thickness. At least one rib is connected on each of the two sides of the backbone portion. Generally, a plurality of ribs are connected to each side of the backbone portion. The number of ribs on each side of the backbone portion can be the same or different. The ribs on each side of the backbone portion can be symmetrically or non-symmetrically spaced apart from one another. The ribs on each side of the backbone portion can have the same or different length, width, shape and/or thickness. The ribs on each side of the backbone portion can be positioned diametrically opposite from one another; however, this is not required. The ribs are generally formed of the same material as the backbone portion; however, this is not required. The ribs are generally bent such that the ends of the ribs are positioned closely together and/or a portion of a plurality of ribs overlap one another. The end of a plurality of ribs can be connected or not connected together. If the end of a plurality of ribs are connected together, the connection is generally a releasable connection that allows the end of the plurality of ribs to separate from one another during the expansion of the medical device. The medical device is generally designed to be expanded by a balloon; however, this is not required. The ends of one or more of the ribs can be curved so that when the medical device is expanded, the ends of the ribs do not penetrate into the inner surface of the body passageway; however, this is not required. Generally, the length of each of the ribs is such that is consists of no more than about 50% of the outer circumference of the medical device in the fully expanded position, typically about 2-48% of the outer circumference of the medical device in the fully expanded position, and more typically about 5-45% of the outer circumference of the medical device in the fully expanded position. In one non-limiting arrangement, the length of a plurality of the ribs is such that is consists of about 20-40% of the outer circumference of the medical device in the fully expanded position.

In one non-limiting aspect of the invention, the medical device can be formed from conventional materials or include new materials having a lower ductility than conventional materials, such as stainless steel or cobalt alloys. In one non-limiting embodiment of the present invention, the medical device is at least partially made of a metal alloy that improves one or more properties (e.g., strength, durability, hardness, biostability, bendability, coefficient of friction, radial strength, flexibility, tensile strength, tensile elongation, longitudinal lengthening, stress-strain properties, improved recoil properties, radiopacity, heat sensitivity, biocompatibility, etc.) of the medical device. The metal alloy that is used to at least partially form the medical device can: 1) increase the radiopacity of the medical device; 2) increase the radial strength of the medical device; 3) increase the yield strength and/or ultimate tensile strength of the medical device; 4) improve the stress-strain properties of the medical device; 5) improve the crimping and/or expansion properties of the medical device; 6) improve the bendability and/or flexibility of the medical device; 7) improve the strength and/or durability of the medical device; 8) increase the hardness of the medical device; 9) improve the longitudinal lengthening properties of the medical device; 10) improve the recoil properties of the medical device; 11) improve the friction coefficient of the medical device; 12) improve the heat sensitivity properties of the medical device; 13) improve the biostability and/or biocompatibility properties of the medical device, 14) enable smaller, thinner and/or lighter weight medical device to be made; and/or 15) resist cracking and/or fatigue. The medical device can be formed by one or more manufacturing processes such as, but are not limited to, laser cutting, electrical discharge machining (EDM), etching, crimping, annealing, drawing, pilgering, electroplating, electro-polishing, chemical polishing, cleaning, pickling, ion beam deposition or implantation, sputter coating, vacuum deposition, wire welding, etc.

In yet another and/or alternative non-limiting aspect of the present invention, a novel metal alloy can be used to form all or a portion of the medical device. In one non-limiting arrangement, the novel alloy includes rhenium and molybdenum. The novel alloy can include one or more other metals such as, but not limited to, calcium, chromium, cobalt, copper, gold, iron, lead, magnesium, nickel, niobium, platinum, rare earth metals, silver, tantalum, titanium, tungsten, yttrium, zinc, zirconium, and/or alloys thereof. In another embodiment of the invention, the medical device can be formed of a biodegradable material (e.g., polymer, metal, etc.). For instance, once the medical device has achieved its function, the medical device can be formed of a material that at least partially dissolves in and/or is at least partially absorbed by the body over time so that the body passageway is eventually free of one or more portions of the stent and/or graft. As such, after the medical device has at least partially fixed the disease or repaired the problem, the medical device can be designed to at least partially dissolve in and/or be at least partially absorbed by the body so that the body passageway is at least partially free of the stent and/or graft. By at least partially removing the medical device from the body passageway, potential problems with thrombosis, in-stent restenosis, vascular narrowing, reoccurrence of vascular malformation, and/or restenosis in the body passageway in and/or around at the treatment location of the medical device is reduced or eliminated. Such removal or partial removal of the medical device from the body passageway also can result in the complete or partial removal of a potential obstruction in the body passageway for potentially future procedures in the body passageway. The biodegradability of one or more portions of the medical device can also fully or partially solve problems associated with fracturing of one or more portions of the device. For instance, when the medical device is in the form of a stent and/or graft that is located in a region subjected to bending (e.g., leg, arms, neck, trachea, etc.), the repeated bending may cause one or more portions of the device to fracture. Over time, one or more fatigued portions of the device may become dislodged from the device. These fractures (e.g., strut and/or graft fractures, etc.) and/or dislodged portions of the device can result in damage to the body passageway and/or one or more regions downstream of the device. The biodegradability of one or more portions of the medical device can facilitate in at least partially overcoming this problem since regions of the medical device prone to fractures and/or dislodgment (i.e., high strain regions) can be formed of a material that at least degrades or is absorbed over time, thus at least partially removing itself from the body passageway of the patient. The biodegradable material is formulated to at least partially dissolve in the body and/or be at least partially absorbed by the body after some period of time (e.g., one month, one year, ten years, etc.) and/or after one or more events (e.g., microfracture, fracture, break, exposure to one or more forms of electromagnetic radiation, exposure to a certain voltage and/or current, exposure to certain sound waves, exposure to certain chemicals and/or agents, etc.). As can be appreciated, the medical device could be at least partially formed of a material that can be caused to dissolve and/or be absorbed in the body and/or cause accelerated rates of dissolving and/or absorption in the body. In such a situation, the device could be caused to begin and/or be caused to accelerate in dissolving and/or bodily absorption so as to at least partially remove the stent, graft, and/or other suitable device, graft, and/or other suitable device from a body passageway to enable another device to be inserted in the body passageway. As can be appreciated, after the other device is inserted, a new device could be reinserted if so needed.

As has been illustrated in these few non-limiting examples, there are numerous applications of the device of the present invention. It will be appreciated that devices other than stents and/or grafts can have many advantages by being partially or fully formed by a biodegradable polymer. For example, experiments have shown limited success in the regeneration of nerve tissue where a nerve has been severed and then attached to either ends of a biodegradable and/or bioabsorbable polymer neurotube. The dissolving and/or bodily absorbing of one or more portions of the medical device can occur naturally and/or be activated and/or controlled by one or more degradation events. In one non-limiting example, the natural dissolving and/or bodily absorbing of one or more portions of the medical device can be achieved by the selection of one or more materials that naturally dissolve and/or are bodily absorbed when inserted in a particular portion of a body. As can be appreciated, one or more coatings can be used to control the time period at which one or more portions of the medical device begin to dissolve and/or be absorbed and/or substantially completely dissolved and/or absorbed; however, this is not required. In another and/or alternative one non-limiting example, one or more portions of the medical device can be caused to dissolve and/or be absorbed and/or accelerate in dissolving and/or being bodily absorbed during and/or after exposure to one or more events. Such degradation events include, but are not limited to, microfracture, fracture, and/or break in one or more portions of the device, exposure of the device to one or more forms of electromagnetic radiation, exposure of the device to one or more forms of particle radiation, exposure of the device to one or more forms of thermal energy, exposure of the device to a certain voltage and/or current, exposure of the device to certain sound waves; exposure of the device to one or more chemicals, and/or exposure of the device to one or more agents delivered in part either systemically and/or locally.

In still another and/or alternative non-limiting aspect of the invention, the medical device includes one or more surface structures or micro-surface structures that are used to facilitate in the operation, function and/or success of the medical device. For example, the one or more surface structures or micro-surface structures can be used to 1) deliver and/or facilitate in the delivery of a biological agent to a treatment area, and/or 2) facilitate in maintaining the medical device in position in a treatment area when the medical device is in an expanded position. The one or more surface structures and/or micro-surface structures can be partially or fully formed of one or more materials that can at least partially dissolve, degrade and/or be absorbed in certain environmental conditions (e.g., exposure to fluids in a body passageway, exposure to enzymes in a body passageway, exposure to air, etc.); however, this is not required. Alternatively, the one or more surface structures and/or micro-surface structures can be partially or fully formed of one or more materials that are biostable and do not dissolve or degrade in a body passageway; however, this is not required.

In another and/or alternative non-limiting aspect of the invention, the one or more surface structures or micro-surface structures on the medical device can be coated with one or more materials; however, this is not required. When the one or more surface structures or micro-surface structures include one or more coatings, such one or more coatings can be used to 1) at least partially control the rate of exposure of the one or more micro-surface structures and/or surface structures to a particular environment (e.g., fluids in a body passageway, gasses in the lungs, bile in a bile duct, air in the surrounding atmosphere, etc.); 2) at least partially control the rate at which one or more micro-surface structures and/or surface structures degrades, dissolves and/or is absorbed; 3) at least partially control the rate at which one or more biological agents are released from the one or more micro-surface structures and/or surface structures; 4) form a smooth coating surface on at least a portion of the one or more micro-surface structures and/or surface structures; 5) form a rough coating surface on at least a portion of the one or more micro-surface structures and/or surface structures; 6) facilitate in one or more of the micro-surface structures and/or surface structures to at least partially secure to, engage with and/or penetrate into a body portion; 7) at least partially shield or protect these one or more micro-surface structures and/or surface structures from damage when the medical device is a) packaged and/or stored, b) unpacked, c) inserted into a treatment area, and/or d) handled by a user; 8) increase the bonding and/or adhesion of one or more biological agents, adhesives, marker materials and/or polymers to the one or more micro-surface structures and/or surface structures; 9) change the appearance or surface characteristics of the medical device; and/or 10) increase the ease that the one or more micro-surface structures and/or surface structures penetrates into a body passageway. As can be appreciated, the one or more coatings of material can be designed and/or formulated to have other and/or additional functions.

In still another and/or alternative non-limiting aspect of the invention, the one or more micro-surface structures and/or surface structures and/or one or more coatings on the micro-surface structures and/or surface structures can be formed of a variety of materials (e.g., metals, polymers, biological agents, adhesives, sugars [e.g., glucose, fructose, sucrose, etc.], carbohydrate compounds, paraffins, starches, salts [e.g., NaCl, etc.], etc.). The one or more materials that form the one or more micro-surface structures and/or surface structures and/or one or more coatings of material can be porous, non-porous, biostable, biodegradable (i.e., dissolves, degrades, is absorbed, or any combination thereof in the body), and/or biocompatible. When one or more polymers are used to at least partially or fully make up the one or more micro-surface structures and/or surface structures and/or one or more coatings of material, the one or more polymers can be porous, non-porous, biostable, biodegradable (i.e., dissolves, degrades, is absorbed, or any combination thereof in the body), and/or biocompatible. When one or more layers of polymer are used to at least partially or fully make up the one or more micro-surface structures and/or surface structures and/or one or more coatings of material, the one or more layers of polymer can be applied by a variety of techniques such as, but not limited to, vapor deposition and/or plasma deposition, spraying, dip-coating, roll coating, sonication, atomization, brushing and/or the like; however, other or additional coating techniques can be used. The one or more polymers can be polymers that are considered to be biodegradable, bioresorbable, or bioerodable; polymers that are considered to be biostable; and/or polymers that can be made to be biodegradable and/or bioresorbable with modification. As can be appreciated, when the body of the medical device is partially or fully formed of biodegradable, bioresorbable, or bioerodable material, the same or similar polymers as the coating can be used. Non-limiting examples of polymers that are considered to be biodegradable, bioresorbable, or bioerodable include, but are not limited to, aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D(lactic acid) and/or copolymers thereof (e.g., DL-PLA), with and without additives (e.g., calcium phosphate glass), and/or other copolymers (e.g., poly(caprolactone lactide), poly(lactide glycolide), poly(lactic acid ethylene glycol)); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethyl ester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/pr copolymers, blends, and/or composites of above. Non-limiting examples of polymers that are considered to be biostable include, but are not limited to, parylene; parylene c; parylene f; parylene n; parylene derivatives; maleic anyhydride polymers; phosphorylcholine; poly n-butyl methacrylate (PBMA); polyethylene-co-vinyl acetate (PEVA); PBMA/PEVA blend or copolymer; polytetrafluoroethene (Teflon®) and derivatives; poly-paraphenylene terephthalamide (Kevlar®); poly(ether ether ketone) (PEEK); poly(styrene-b-isobutylene-b-styrene) (Translute™); tetramethyldisiloxane (side chain or copolymer); polyimides polysulfides; poly(ethylene terephthalate); poly(methyl methacrylate); poly(ethylene-co-methyl methacrylate); styrene-ethylene/butylene-styrene block copolymers; ABS; SAN; acrylic polymers and/or copolymers (e.g., n-butyl-acrylate, n-butyl methacrylate, 2-ethylhexyl acrylate, lauryl-acrylate, 2-hydroxy-propyl acrylate, polyhydroxyethyl, methacrylate/methylmethacrylate copolymers); glycosaminoglycans; alkyd resins; elastin; polyether sulfones; epoxy resin; poly(oxymethylene); polyolefins; polymers of silicone; polymers of methane; polyisobutylene; ethylene-alphaolefin copolymers; polyethylene; polyacrylonitrile; fluorosilicones; poly(propylene oxide); polyvinyl aromatics (e.g., polystyrene); poly(vinyl ethers) (e.g., polyvinyl methyl ether); poly(vinyl ketones); poly(vinylidene halides) (e.g., polyvinylidene fluoride, polyvinylidene chloride); poly(vinylpyrolidone); poly(vinylpyrolidone)/vinyl acetate copolymer; polyvinylpridine prolastin or silk-elastin polymers (SELP); silicone; silicone rubber; polyurethanes (polycarbonate polyurethanes, silicone urethane polymer) (e.g., chronoflex varieties, bionate varieties); vinyl halide polymers and/or copolymers (e.g., polyvinyl chloride); polyacrylic acid; ethylene acrylic acid copolymer; ethylene vinyl acetate copolymer; polyvinyl alcohol; poly(hydroxyl alkylmethacrylate); polyvinyl esters (e.g., polyvinyl acetate); and/or copolymers, blends, and/or composites of above. Non-limiting examples of polymers that can be made to be biodegradable and/or bioresorbable with modification include, but are not limited to, hyaluronic acid (hyanluron); polycarbonates; polyorthocarbonates; copolymers of vinyl monomers; polyacetals; biodegradable polyurethanes; polyacrylamide; polyisocyanates; polyamide; and/or copolymers, blends, and/or composites of above. As can be appreciated, other and/or additional polymers and/or derivatives of one or more of the above listed polymers can be used for the one or more coatings of material.

In yet another and/or alternative non-limiting aspect of the invention, the one or more micro-surface structures and/or surface structures and/or one or more coatings on the micro-surface structures and/or surface structures can be coated with and/or fully be formed of or partially include one or more biological agents. The term “biological agent” includes, but is not limited to, a substance, drug, etc. formulated and/or designed to prevent, inhibit and/or treat one or more biological problems, and/or to promote the healing in a treated area. The following categories of biological agents can be used: thrombolytics, vasodilators, anti-hypertensive agents, anti-microbial or anti-biotic, anti-mitotic, anti-proliferative, anti-secretory agents, non-steroidal anti-inflammatory drugs, immunosuppressive agents, growth factors and growth factor antagonists, chemotherapeutic agents, anti-polymerases, anti-viral agents, anti-body targeted therapy agents, hormones, anti-oxidants, radio-therapeutic agents, radiopaque agents and/or radio-labeled agents. As can be appreciated, other or additional types of biological agents can be used. Non-limiting examples of biological problems that can be addressed by one or more biological agents include, but are not limited to, viral, fungal and/or bacterial infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; and/or the like. Non-limiting examples of biological agents that can be used include, but are not limited to, 5-Fluorouracil and/or derivatives thereof; 5-Phenylmethimazole and/or derivatives thereof; ACE inhibitors and/or derivatives thereof; acenocoumarol and/or derivatives thereof; acyclovir and/or derivatives thereof; actilyse and/or derivatives thereof; adrenocorticotropic hormone and/or derivatives thereof; adriamycin and/or derivatives thereof; agents that modulate intracellular Ca₂₊ transport such as L-type (e.g., diltiazem, nifedipine, verapamil, etc.) or T-type Ca₂₊ channel blockers (e.g., amiloride, etc.); alpha-adrenergic blocking agents and/or derivatives thereof; alteplase and/or derivatives thereof; amino glycosides and/or derivatives thereof (e.g., gentamycin, tobramycin, etc.); angiopeptin and/or derivatives thereof; angiostatic steroid and/or derivatives thereof; angiotensin II receptor antagonists and/or derivatives thereof; anistreplase and/or derivatives thereof; antagonists of vascular epithelial growth factor and/or derivatives thereof; anti-biotics; anti-coagulant compounds and/or derivatives thereof; anti-fibrosis compounds and/or derivatives thereof; anti-fungal compounds and/or derivatives thereof; anti-inflammatory compounds and/or derivatives thereof; anti-invasive factor and/or derivatives thereof; anti-metabolite compounds and/or derivatives thereof (e.g., staurosporin, trichothecenes, and modified diphtheria and ricin toxins, Pseudomonas exotoxin, etc.); anti-matrix compounds and/or derivatives thereof (e.g., colchicine, tamoxifen, etc.); anti-microbial agents and/or derivatives thereof; anti-migratory agents and/or derivatives thereof (e.g., caffeic acid derivatives, nilvadipine, etc.); anti-mitotic compounds and/or derivatives thereof; anti-neoplastic compounds and/or derivatives thereof; anti-oxidants and/or derivatives thereof; anti-platelet compounds and/or derivatives thereof; anti-proliferative compounds and/or derivatives thereof; anti-thrombogenic agents and/or derivatives thereof; argatroban and/or derivatives thereof; ap-1 inhibitors and/or derivatives thereof (e.g., for tyrosine kinase, protein kinase C, myosin light chain kinase, Ca₂₊/calmodulin kinase II, casein kinase II, etc.); aspirin and/or derivatives thereof; azathioprine and/or derivatives thereof; β-Estradiol and/or derivatives thereof; β-1-anticollagenase and/or derivatives thereof; calcium channel blockers and/or derivatives thereof; calmodulin antagonists and/or derivatives thereof (e.g., H₇, etc.); CAPTOPRIL and/or derivatives thereof; cartilage-derived inhibitor and/or derivatives thereof; ChIMP-3 and/or derivatives thereof; cephalosporin and/or derivatives thereof (e.g., cefadroxil, cefazolin, cefaclor, etc.); chloroquine and/or derivatives thereof; chemotherapeutic compounds and/or derivatives thereof (e.g., 5-fluorouracil, vincristine, vinblastine, cisplatin, doxyrubicin, adriamycin, tamocifen, etc.); chymostatin and/or derivatives thereof; CILAZAPRIL and/or derivatives thereof; clopidigrel and/or derivatives thereof; clotrimazole and/or derivatives thereof; colchicine and/or derivatives thereof; cortisone and/or derivatives thereof; coumadin and/or derivatives thereof; curacin-A and/or derivatives thereof; cyclosporine and/or derivatives thereof; cytochalasin and/or derivatives thereof (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.); cytokines and/or derivatives thereof; desirudin and/or derivatives thereof; dexamethazone and/or derivatives thereof; dipyridamole and/or derivatives thereof; eminase and/or derivatives thereof; endothelin and/or derivatives thereof; endothelial growth factor and/or derivatives thereof; epidermal growth factor and/or derivatives thereof; epothilone and/or derivatives thereof; estramustine and/or derivatives thereof; estrogen and/or derivatives thereof; fenoprofen and/or derivatives thereof; fluorouracil and/or derivatives thereof; flucytosine and/or derivatives thereof; forskolin and/or derivatives thereof; ganciclovir and/or derivatives thereof; glucocorticoids and/or derivatives thereof (e.g., dexamethasone, betamethasone, etc.); glycoprotein IIb/IIIa platelet membrane receptor antibody and/or derivatives thereof; GM-CSF and/or derivatives thereof; griseofulvin and/or derivatives thereof; growth factors and/or derivatives thereof (e.g., VEGF; TGF; IGF; PDGF; FGF, etc.); growth hormone and/or derivatives thereof; heparin and/or derivatives thereof; hirudin and/or derivatives thereof; hyaluronate and/or derivatives thereof; hydrocortisone and/or derivatives thereof; ibuprofen and/or derivatives thereof; immunosuppressive agents and/or derivatives thereof (e.g., adrenocorticosteroids, cyclosporine, etc.); indomethacin and/or derivatives thereof; inhibitors of the sodium/calcium antiporter and/or derivatives thereof (e.g., amiloride, etc.); inhibitors of the IP₃ receptor and/or derivatives thereof; inhibitors of the sodium/hydrogen antiporter and/or derivatives thereof (e.g., amiloride and derivatives thereof; etc.); insulin and/or derivatives thereof; interferon alpha-2-macroglobulin and/or derivatives thereof; ketoconazole and/or derivatives thereof; Lepirudin and/or derivatives thereof; lisinipril and/or derivatives thereof; lovastatin and/or derivatives thereof; marevan and/or derivatives thereof; mefloquine and/or derivatives thereof; metalloproteinase inhibitors and/or derivatives thereof; methotrexate and/or derivatives thereof; metronidazole and/or derivatives thereof; miconazole and/or derivatives thereof; monoclonal antibodies and/or derivatives thereof; mutamycin and/or derivatives thereof; naproxen and/or derivatives thereof; nitric oxide and/or derivatives thereof; nitroprusside and/or derivatives thereof; nucleic acid analogues and/or derivatives thereof (e.g., peptide nucleic acids, etc.); nystatin and/or derivatives thereof; oligonucleotides and/or derivatives thereof; paclitaxel and/or derivatives thereof; penicillin and/or derivatives thereof; pentamidine isethionate and/or derivatives thereof; phenindione and/or derivatives thereof; phenylbutazone and/or derivatives thereof; phosphodiesterase inhibitors and/or derivatives thereof; plasminogen activator inhibitor-1 and/or derivatives thereof; plasminogen activator inhibitor-2 and/or derivatives thereof; platelet factor 4 and/or derivatives thereof; platelet derived growth factor and/or derivatives thereof; plavix and/or derivatives thereof; POSTMI 75 and/or derivatives thereof; prednisone and/or derivatives thereof; prednisolone and/or derivatives thereof; probucol and/or derivatives thereof; progesterone and/or derivatives thereof; prostacyclin and/or derivatives thereof; prostaglandin inhibitors and/or derivatives thereof; protamine and/or derivatives thereof; protease and/or derivatives thereof; protein kinase inhibitors and/or derivatives thereof (e.g., staurosporin, etc.); quinine and/or derivatives thereof; radioactive agents and/or derivatives thereof (e.g., Cu-64, Ca-67, Cs-131, Ga-68, Zr-89, Ku-97, Tc-99m, Rh-105, Pd-103, Pd-109, In-111, 1-123, 1-125, 1-131, Re-186, Re-188, Au-198, Au-199, Pb-203, At-211, Pb-212, Bi-212, H₃P³²O₄, etc.); rapamycin and/or derivatives thereof; receptor antagonists for histamine and/or derivatives thereof; refludan and/or derivatives thereof; retinoic acids and/or derivatives thereof; revasc and/or derivatives thereof; rifamycin and/or derivatives thereof; sense or anti-sense oligonucleotides and/or derivatives thereof (e.g., DNA, RNA, plasmid DNA, plasmid RNA, etc.); seramin and/or derivatives thereof; steroids; seramin and/or derivatives thereof; serotonin and/or derivatives thereof; serotonin blockers and/or derivatives thereof; streptokinase and/or derivatives thereof; sulfasalazine and/or derivatives thereof; sulfonamides and/or derivatives thereof (e.g., sulfamethoxazole, etc.); sulphated chitin derivatives; sulphated polysaccharide peptidoglycan complex and/or derivatives thereof; T_{H1} and/or derivatives thereof (e.g., Interleukins-2, -12, and -15, gamma interferon, etc.); thioprotese inhibitors and/or derivatives thereof; taxol and/or derivatives thereof (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.); ticlid and/or derivatives thereof; ticlopidine and/or derivatives thereof; tick anti-coagulant peptide and/or derivatives thereof; thioprotese inhibitors and/or derivatives thereof; thyroid hormone and/or derivatives thereof; tissue inhibitor of metalloproteinase-1 and/or derivatives thereof; tissue inhibitor of metalloproteinase-2 and/or derivatives thereof; tissue plasma activators; TNF and/or derivatives thereof, tocopherol and/or derivatives thereof; toxins and/or derivatives thereof; tranilast and/or derivatives thereof; transforming growth factors alpha and beta and/or derivatives thereof; trapidil and/or derivatives thereof; triazolopyrimidine and/or derivatives thereof; vapiprost and/or derivatives thereof; vinblastine and/or derivatives thereof; vincristine and/or derivatives thereof; zidovudine and/or derivatives thereof As can be appreciated, the biological agent can include one or more derivatives of the above listed compounds and/or other compounds. As can also be appreciated, two or more different biological agents can be used. Typically, the amount of biological agent included on, in, and/or used in conjunction with the one or more micro-surface structures and/or surface structures and/or one or more coatings on the micro-surface structures and/or surface structures is about 0.01-100 ug per mm²; however, other amounts can be used. The amount of two or more biological agents on, in, and/or used in conjunction with the medical device can be the same or different. The one or more biological agents can be coated on and/or impregnated in the one or more micro-surface structures and/or surface structures and/or one or more coatings on the micro-surface structures and/or surface structures by a variety of mechanisms such as, but not limited to, spraying (e.g., atomizing spray techniques, etc.), dip coating, roll coating, sonication, brushing, plasma deposition, depositing by vapor deposition. As can be appreciated, when the one or more micro-surface structures and/or surface structures include one or more biological agents, the one or more micro-surface structures and/or surface structures can be partially or fully grown from the one or more biological agents.

In still yet another and/or alternative non-limiting aspect of the invention, the medical device of the present invention can include, but is not limited to, stents, grafts, and vascular grafts. In one non-limiting embodiment of the invention, the medical device is directed for use in a body passageway. As defined herein, the term “body passageway” is defined herein to be any passageway or cavity in a living organism (e.g., bile duct, bronchiole tubes, nasal cavity, blood vessels, heart, esophagus, trachea, stomach, fallopian tube, uterus, ureter, urethra, the intestines, lymphatic vessels, nasal passageways, eustachian tube, acoustic meatus, etc.). For vascular applications, the term “body passageway” primarily refers to blood vessels and chambers in the heart. When the medical device is in the form of a stent, the stent can be an expandable stent that is expandable by an inflatable device (e.g., balloon, etc.) and/or other means. The medical device can be at least partially 1) a biodegradable device that at least partially dissolves in the body and/or is absorbed by the body, and/or 2) a biostable device that resists or does not dissolve in the body and/or is absorbed by the body. The medical device is typically made of a material that imparts the desirable mechanical properties to the medical device (e.g., strength, durability, expandability, flexibility, hardness, biostability, bendability, coefficient of friction, radial strength, flexibility, tensile strength, tensile elongation, longitudinal lengthening, stress-strain properties, improved recoil properties, radiopacity, heat sensitivity, biocompatibility, etc.). When the medical device is in the form of a stent, the stent can be designed to be insertable into a treatment area in a body passageway and expand in the treatment area.

In a further and/or alternative non-limiting aspect of the present invention, the one or more micro-surface structures and/or surface structures on the medical device can be constructed by use of one or more microelectromechanical manufacturing (MEMS) techniques (e.g., micro-machining, laser micro-machining, laser micro-machining, micro-molding, etc.); however, other or additional manufacturing techniques can be used. The one or more micro-surface structures can include one or more micro-needles, micro-cylinders, micro-cones, micro-pyramids, micro-parallelopipes, micro-prisms, micro-hemispheres, etc. on the surface of the medical device. As defined herein, a micro-surface structure is a structure that has at least one dimension (e.g., average width, average diameter, average height, average length, average depth, etc.) that is no more than about 2 mm, and typically no more than about 1 mm. Surface structures are structures that are defined as being larger than micro-surface structures. Typically, the micro-surface structures extend from the outer surface no more than about 400 microns, and more typically, less than about 300 microns, and more typically about 15-250 microns; however, other sizes can be used. The micro-surface structures can be clustered together or disbursed throughout the surface of the medical device. Similar shaped and/or sized micro-surface structures can be used, or different shaped and/or sized micro-surface structures can be used. The one or more micro-surface structures can be designed to contain and/or be fluidly connected to a passageway, cavity, etc. in the medical device; however, this is not required. The one or more micro-surface structures can be used to engage and/or penetrate surrounding tissue or organs once the medical device has been positioned on and/or in a patient; however, this is not required. One or more of the micro-surface structures can include one or more internal passageways that can include one or more materials (e.g., biological agent, polymer, etc.); however, this is not required. The one or more micro-surface structures can be formed by a variety of processes (e.g., machining, chemical modifications, chemical reactions, MEMS (e.g., micro-machining, etc.), etching, laser cutting, etc.). The one or more micro-surface structures can be biostable, biodegradable, etc.

In still a further and/or alternative aspect of the present invention, the medical device can be coated with one or more biological agents. A non-limiting list of one or more biological agents that can be coated on the medical device have been previously listed above. The one or more biological agents, when used on the medical device, can be released in a controlled manner; however, this is not required. As can be appreciated, controlled release of one or more biological agents on the medical device is not always required and/or desirable. As such, one or more of the biological agents on and/or in the medical device can be uncontrollably released from the medical device during and/or after insertion of the medical device in the treatment area. It can also be appreciated that one or more biological agents on the medical device can be controllably released from the medical device and one or more biological agents on the medical device can be uncontrollably released from the medical device. As such, the medical device can be designed such that 1) all the biological agent on the medical device is controllably released, 2) some of the biological agent on the medical device is controllably released and some of the biological agent on the medical device is non-controllably released, or 3) none of the biological agent on the medical device is controllably released. The medical device can also be designed such that the rate of release of the one or more biological agents from the medical device is the same or different. Non-limiting arrangements that can be used to control the release of one or more biological agent from the medical device include a) at least partially coating one or more biological agents with one or more polymers, b) at least partially incorporating and/or at least partially encapsulating one or more biological agents into and/or with one or more polymers, and/or c) inserting one or more biological agents in pores, passageways, cavities, etc. in the medical device and at least partially coat or cover such pores, passageways, cavities, etc. with one or more polymers and/or micro-surface structures. The one or more polymers used to at least partially control the release of one or more biological agent from the medical device can be porous or non-porous. The one or more biological agents can be inserted into and/or applied to one or more micro-surface structures on the medical device, and/or be used to at least partially form one or more micro-surface structures on the medical device. As such, the one or more biological agents on the medical device can be 1) coated on one or more surface regions of the medical device; 2) inserted and/or impregnated in one or more micro-surface structures and/or surface structures, etc. of the medical device;, and/or 3) form at least a portion or be included in at least a portion of the one or more micro-surface structures and/or surface structures of the medical device. When one or more biological agents are used on the medical device, the one or more biological agents can be 1) directly coated on one or more surfaces of the medical device; 2) mixed with one or more coating polymers or other coating materials and then at least partially coated on one or more surfaces of the medical device; 3) at least partially coated on the surface of another coating material that has been at least partially coated on the medical device; and/or 4) at least partially encapsulated between a) a surface or region of the medical device and one or more other coating materials and/or b) two or more other coating materials. As can be appreciated, many other coating arrangements can be additionally or alternatively used. When the one or more biological agents are inserted and/or impregnated in one or more micro-surface structures and/or surface structures of the medical device, 1) one or more other coating materials can be applied at least partially over the one or more micro-surface structures and/or surface structures of the medical device; and/or 2) one or more polymers can be combined with one or more biological agents. As such, the one or more biological agents can be 1) embedded in the structure of the medical device; 2) positioned in one or more micro-surface structures and/or surface structures of the medical device; 3) encapsulated between two polymer coatings; 4) encapsulated between the base structure and a polymer coating and/or protective coating; 5) mixed in the base structure of the medical device that includes at least one polymer coating; or 6) one or more combinations of 1, 2, 3, 4 and/or 5. In addition or alternatively, the one or more coatings of the one or more polymers on the medical device can include 1) one or more coatings of non-porous polymers; 2) one or more coatings of a combination of one or more porous polymers and one or more non-porous polymers; 3) one or more coatings of porous polymer; or 4) one or more combinations of options 1, 2, and 3. As can be appreciated different biological agents can be located in and/or between different polymer coating layers and/or on the structure of the medical device. As can also be appreciated, many other and/or additional coating combinations and/or configurations can be used. The concentration of one or more biological agents, the type of polymer, the type of material coating, the type and/or shape of internal structures in the medical device and/or the coating thickness of one or more biological agents can be used to control the release time, the release rate and/or the dosage amount of one or more biological agents; however, other or additional combinations can be used. As such, the biological agent and polymer system combination and location on the medical device can be numerous. As can also be appreciated, one or more biological agents can be deposited on the top surface of the medical device to provide an initial uncontrolled burst effect of the one or more biological agents prior to 1) the controlled release of the one or more biological agents through one or more layers of polymer system and/or 2) the uncontrolled release of the one or more biological agents through one or more layers of polymer system; however, this is not required.

In still another and/or alternative non-limiting aspect of the invention, the medical device can optionally include a protective material that is used to fully or partially cover, embed, and/or coat the one or more of the micro-surface structures and/or surface structures on the medical device. The protective coating generally is coated on both the outer surface of the medical device and the micro-surface structures and/or surface structures on the medical device. The coating thickness of the protective material can be uniform; however, this is not required. For instance, when the protective coating is on both the outer surface of the medical device and the micro-surface structures and/or surface structures, the coating thickness on the outer surface of the medical device may be generally uniform and the coating thickness on the various regions of the micro-surface structures and/or surface structures (e.g., top, top portion, base, bottom portion, middle portion. etc.) can vary. Specifically, when the protective material is applied on the outer surface of the medical device to be generally uniform, since the micro-surface structures extend upwardly from the outer surface, the base of the micro-surface structures will generally have a thicker coating of protective material than the top portion of the micro-surface structures. Generally, the protective material is formed of a different material than the outer surface of the medical device, and/or the micro-surface structures and/or surface structures on the medical device; however, this is not required. Non-limiting examples of materials that can be used to fully or partially form the protective material are polymers, biological agents, adhesives, sugars [e.g., glucose, fructose, sucrose, etc.], carbohydrate compounds, paraffins, starches, salts [e.g., NaCl, etc.], etc. The protective material can optionally include one or more biological agents. The protective material is generally formed of a material that will dissolve and/or degrade faster than the outer surface of the medical device, and/or the micro-surface structures and/or surface structures on the medical device so as to rapidly expose or more fully expose the outer surface of the medical device and the micro-surface structures and/or surface structures on the medical device when the medical device is positioned in the treatment area of the body passageway; however, this is not required.

In one non-limiting embodiment of the invention, the one or more coatings of protective material generally follow the profile of the one or more of the micro-surface structures and/or surface structures on the medical device; however, this is not required. In another non-limiting embodiment of the invention, the one or more coatings of protective material have a generally uniform thickness on the outer surface of the medical device, which thickness may be less than equal to or greater than the height of the surface structures and/or micro-surface structures so that such surface structures and/or micro-surface structures are partially or fully embedded under the protective material. In one non-limiting aspect of this embodiment, the coating thickness of the protective material is generally at least about 0.001 μm and typically less than about 500 μm; however, other thickness can be used depending on the size and/or shape of the one or more of the micro-surface structures and/or surface structures on the medical device. In another and/or alternative aspect of this embodiment, the thickness of the one or more coatings of protective material form a coating layer that is about 0.01-150 μm, and typically about 0.1-50 μm; however, it will be appreciated that other thicknesses can be used. In another and/or alternative embodiment of the invention, the one or more coatings of protective material form a coating layer that partially or fully embeds one or more of the micro-surface structures and/or surface structures on the medical device within the one or more coatings of protective material. The coating thickness of such a coating typically depends on the size and/or shape of the one or more of the micro-surface structures, and/or surface structures on the medical device, and/or whether the one or more coatings of protective material are to be partially or fully embedded within the one or more coatings of protective material.

In yet a further and/or alternative non-limiting aspect of the invention, the medical device can include a marker material that facilitates in enabling the medical device to be properly positioned in a body passageway. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). In one non-limiting embodiment, the marker material is visible to x-rays (i.e., radiopaque). The marker material can form all or a portion of the medical device and/or be coated on one or more portions (flaring portion and/or body portion at ends of medical device, at or near transition of body portion and flaring section, etc.) of the medical device. The location of the marker material can be on one or multiple locations on the medical device. The size of the one or more regions that include the marker material can be the same or different. The marker material can be spaced at defined distances from one another so as to form ruler-like markings on the medical device to facilitate in the positioning of the medical device in a body passageway. The marker material can be a rigid or flexible material. The marker material can be a biostable or biodegradable material.

In another and/or alternative non-limiting aspect of the present invention, the medical device is designed to improve patient procedural outcome. The medical device can be designed to be used as a biological agent delivery mechanism to deliver one or more biological agents to and/or into a wall of a body passageway and/or downstream from the site of implantation of the medical device. In one non-limiting embodiment of the invention, the medical device is designed to deliver one or more biological agents directly into the wall of a body passageway. In another and/or alternative non-limiting embodiment of the invention, the medical device is designed to at least partially utilize molecular diffusion to deliver one or more biological agents to and/or into a wall of a body passageway and/or downstream from the site of implantation of the medical device; however, this is not required. When a molecular diffusion mechanism is used, this mechanism can be used to at least partially control the diffusion of one or more biological agents from the medical device. When a molecular diffusion mechanism is used on the medical device, one or more non-porous polymer layers can be used to facilitate in such molecular diffusion; however, this is not required. In still another and/or alternative non-limiting embodiment of the invention, the medical device includes one or more micro-surface structures on the outer surface of the inflatable device. The medical device can include at least one layer of biological agent and/or at least one polymer coating on the outer surface of the inflatable device; however, this is not required. The one or more micro-surface structures on the surface of the medical device can be partially formed and/or coated with one or more layers of biological agent and/or polymer coating; however, this is not required.

In still another and/or alternative non-limiting aspect of the present invention, the medical device includes one or more protrusions (e.g., needles, micro-needles, etc.) on the outer surface of the medical device. These one or more protrusions are generally biodegradable; however, this is not required. These one or more protrusions generally include or are formed of one or more biological agents. These one or more protrusions are designed to at least partially penetrate into the surface of a body passageway when the medical device is expanded in the body passageway. These one or more protrusions can optionally be designed to break-off of the medical device after the medical device has been expanded in the body passageway and after the one or more protrusions have at least partially penetrated in the body passageway; however, this is not required. These one or more protrusions can optionally be designed to enable fluid (e.g., biological agent, etc.) to flow from the medical device, at least partially through the protrusions and into the body passageway after the medical device has been expanded in the body passageway and after the one or more protrusions have at least partially penetrated in the body passageway; however, this is not required. Additional non-limiting features of the invention are disclosed in the attached drawings. Additional information regarding the one or more protrusions and formation of the one or more protrusions is disclosed in U.S. Patent Application Nos. 2006/0224237; 2006/0198869; 2007/0123973; 2006/0200226; and US2010/0096778, which are incorporated herein by reference.

In yet another and/or alternative non-limiting aspect of the present invention, the medical device includes one or more protrusions on the outer surface of the medical device that are designed to at least partially penetrate into the wall of a body passageway (e.g., blood vessel, etc.) when the medical device is expanded in the treatment area of the body passageway. As can be appreciated, none, a portion, or all of the protrusions can be formed of a biodegradable and/or bioabsorbable material. As can also be appreciated, a portion or all of the protrusions on the outer surface of the medical device can be designed to at least partially penetrate into the wall of a body passageway. In one non-limiting embodiment of the invention, the top portion of the one or more protrusions can include a tapered or sharpened region to facilitate in the penetration of the one or more protrusions into the wall of the body passageway; however, this is not required. The one or more protrusions can be formed in a variety of ways (e.g., molding, etching, growing, laminating, stamping, MEMS processes, lithographic techniques, inkjet technology, etc.). The size and/or shape of the one or more protrusions can be the same or different. The distribution of a plurality of protrusions on the outer surface of the medical device can be uniform or non-uniform. As can be appreciated, certain patterns of protrusions can be formed on the outer surface of the medical device. As can be appreciated, a portion or all of the protrusions can include a tapered or sharpened region to facilitate in the penetration of the one or more protrusions into the wall of the body passageway. The shape of the tapered or sharpened region on the one or more protrusions, when used, is non-limiting. In one non-limiting configuration, the shape of the tapered or sharpened region on the one or more protrusions is similar to a needle point. In another non-limiting configuration, the shape of the tapered or sharpened top region on the one or more protrusions is similar to an arrow head. In still non-limiting configuration, the shape of the tapered or sharpened top region on the one or more protrusions is designed to penetrate into the wall of a body passageway, but resist being removed from the body passageway once partial or full penetration of the body passageway has occurred. In another and/or alternative non-limiting embodiment of the invention, all or a portion of the one or more protrusions can be designed to break off from the remaining body of the one or more protrusions and/or from the outer surface of the medical device. When the one or more protrusions includes and/or is at least partially formed of one or more biological agents, the portion of the one or more protrusions remaining embedded in the body passageway can be used to continue to locally supply biological agent to the treatment area of the body passageway. As can be appreciated, a portion or all of the protrusions include and/or are partially or fully formed of biological agent. As can also be appreciated, a portion or all of the protrusions may be absent biological agent. In one non-limiting configuration, the shape of the top region on the one or more protrusions can be designed to break off from the remaining body of the one or more protrusions. The one or more protrusions can include one or more notches, narrowed regions, etc. to facilitate in the breaking off of the tapered or sharpened top region from the remaining body of the one or more protrusions. In another and/or alternative non-limiting configuration, the body of the one or more protrusions can be designed to break off from the remaining body of the one or more protrusions, and/or the body of the one or more protrusions can be designed to break off from the outer surface of the medical device during or after the one or more protrusions have partially or fully penetrated into the wall of the body passageway. The one or more protrusions can include one or more notches, narrowed regions, etc. to facilitate in the breaking off of the body from another portion of the body and/or the outer surface of the medical device; however, this is not required. Also, or alternatively, the one or more protrusions can be formed of different materials in different regions of the protrusions, wherein the type of materials and/or the regions the materials are used in facilitate in the breaking of the protrusion at a certain location or region; however, this is not required.

In one non-limiting overview of the present invention, there is provided a medical device that is adapted for introduction into a patient. The medical device can be designed to include and/or to release one or more biological agents in a controlled and/or uncontrolled fashion; however, this is not required. For instance, all of the biological agent can be controllably released from the medical device, all of the biological agent can be uncontrollably released from the medical device, or some of the biological agent can be controllably released and some uncontrollably released from the medical device. The controlled release of the one or more biological agents can be by molecular diffusion through one or more polymer layers; however, it will be appreciated that other or additional mechanisms can be used to control the rate of release. The polymer and/or biological agent that is included on and/or forms at least a portion of the medical device can be hydrophobic or hydrophilic, which can be used to facilitate in the controlled release of the one or more biological agents; however, this is not required. The thickness of the one or more polymer layers can be selected to facilitate in the controlled release of the one or more biological agents; however, this is not required. The molecular weight and/or molecular structure of the one or more biological agents and/or one or more polymers can be selected to facilitate in the release of the one or more biological agents; however, this is not required. The medical device can have a variety of applications such as, but not limited to, placement into the vascular system, esophagus, trachea, colon, biliary tract, or urinary tract.

As can be appreciated, the medical device can have other or additional uses. The medical device can include one or more layers of polymer and/or biological agent on the surface structure of the medical device; however, this is not required. At least one biological agent can be deposited underneath and/or combined with at least one non-porous polymer layer so as to control the release of the at least one biological agent; however, this is not required. The medical device can also include one or more porous polymers; however, this is not required. One or more polymer layers, when used, can be applied by vapor deposition, plasma deposition, or another or additional coating technique. The medical device can be in the form of a stent. The medical device can include one or more surface structures and/or micro-surface structures that can include and/or be at least partially formed by one or more biological agents and/or polymers. These structures can be at least partially formed by MEMS (e.g., micro-machining, etc.) technology, layer growing processes, and/or other types of technology. The structures can be designed to contain and/or be fluidly connected to a passageway in the medical device; however, this is not required. The micro-surface structures can be designed to engage and/or penetrate surrounding tissue or organs once the medical device has been positioned on and/or in a patient and inflated; however, this is not required. One or more polymers and/or biological agents can be inserted in these surface structures and/or at least partially form these surface structures and/or micro-surface structures of the medical device; however, this is not required. Typically, the micro-surface structures, when formed, extend from or into the outer surface no more than about 400 microns, and more typically less than about 300 microns, and more typically about 15-250 microns; however, other sizes can be used. The micro-surface structures can be clustered together or disbursed throughout the surface of the medical device. Similar shaped and/or sized micro-surface structures can be used, or different shaped and/or sized micro-surface structures can be used. The surface topography of the medical device can be uniform or vary to achieve the desired operation and/or biological agent released from the medical device. As can be appreciated, the medical device or one or more regions of the medical device can be constructed by use of one or more MEMS techniques (e.g., micro-machining, etc.), layer growing techniques, molding techniques; however, this is not required. As can be appreciated one or more biological agents and/or polymers can be placed on different regions of the medical device to achieve the desired operation and/or biological agent released from the medical device. The medical device can be used in conjunction with other biological agents. The introduction of biological agents from a source other than the medical device can have a synergistic effect which can enhance the success of the medical device. The medical device of the present invention can be used to provide localized delivery of a therapeutic agent in both de-novo lesions and for in-stent restenosis. The medical device can include one or more protrusions (e.g., needles, etc.) that are able to penetrate the vasculature and optionally detach, leaving behind a portion that is made of or includes a polymer/biological agent. A bolus of biological agent can also be provided during such procedure. The biological agent can be formulated to relieve restenosis, thrombus formation, inflammation, etc.; however, this is not required.

One non-limiting object of the present invention is the provision of a medical device that includes a backbone portion and one or more rib portions.

Another and/or alternative non-limiting object of the present invention is the provision of a medical device that has a discontinuous circumference when expanded in a body passageway.

Yet another and/or alternative non-limiting object of the present invention is the provision of a medical device that includes an inflatable device that includes one or more surface structures or micro-surface structures.

Another and/or alternative non-limiting object of the present invention is the provision of a medical device that includes one or more surface structures or micro-surface structures, which one or more surface structures or micro-surface structures include and/or are at least partially formed of one or more biological agents and/or one or more polymers.

Still another and/or alternative non-limiting object of the present invention is the provision of a medical device that includes one or more surface structures or micro-surface structures, which one or more surface structures or micro-surface structures are designed to at least partially penetrate into a body passageway.

Yet another and/or alternative non-limiting object of the present invention is the provision of a medical device that includes one or more surface structures or micro-surface structures, which one or more surface structures or micro-surface structures are designed to at least partially penetrate into a body passageway and at least a portion of the surface structures or micro-surface structures is to break-off or detach from another portion of the surface structures or micro-surface structures or detach from the inflatable device and then remain in the penetrated wall of the body passageway.

Still yet another and/or alternative non-limiting object of the present invention is the provision of a medical device that includes one or more surface structures or micro-surface structures, which one or more surface structures or micro-surface structures are designed to at least partially penetrate into a body passageway and locally deliver one or more biological agents in a treatment area.

Another and/or alternative non-limiting object of the present invention is the provision of a medical device that has a generally C-shaped cross-section shape when expanded.

These and other advantages will become apparent to those skilled in the art upon the reading and following of this description taken together with the accompanying drawings.

### BRIEF DESCRIPTION OF THE DRAWINGS

Reference may now be made to the drawings, which illustrate various embodiments that the invention may take in physical form and in certain parts and arrangements of parts wherein:

FIG. 1 is a front view of one the expanded position e non-limiting the medical device of the present invention in accordance with the present invention;

FIG. 2 is a top view of a non-limiting backbone of the medical device;

FIG. 3 is a top view of a non-limiting the medical device prior to the ribs being bent to form the medical device in the non-expanded position;

FIG. 4 is another top view of a non-limiting the medical device prior to the ribs being bent to form the medical device in the non-expanded position;

FIG. 5 is another top view of a non-limiting the medical device prior to the ribs being bent to form the medical device in the non-expanded position;

FIG. 6 is an end view of a non-limiting medical device in the formed non-expanded position;

FIG. 7 is an end view of the medical device of FIG. 6 in the expanded position;

FIG. 8 is an end view of another non-limiting medical device in the formed non-expanded position; and,

FIG. 9 is an end view of the medical device of FIG. 8 is the expanded position.

### DETAILED DESCRIPTION OF THE INVENTION

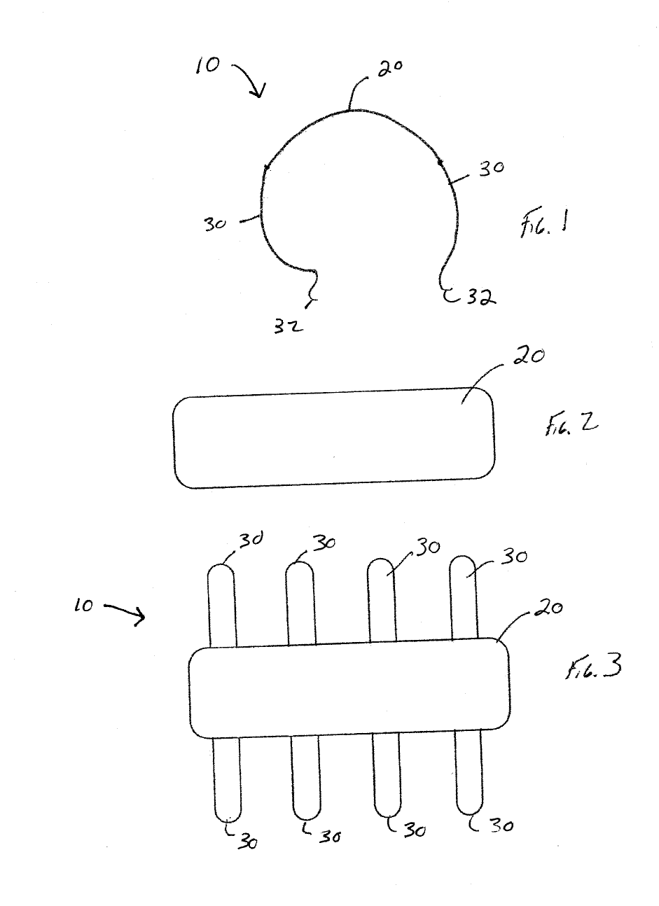

Referring now to the drawings wherein the showings are for the purpose of illustrating embodiments of the invention only and not for the purpose of limiting the same, FIGS. 1-9 illustrate non-limiting embodiments of the medical device 10 in accordance with the present invention.

Referring now to FIG. 3, there is illustrated one non-limiting configuration of the medical device 10 in accordance with the present invention. The medical device 10 includes a backbone portion 20 and a plurality of ribs 30 that are connected to the backbone portion. The backbone portion and ribs can be formed from a single piece of material such that the ribs are integrally formed with the backbone portion; however, this is not required. One or more ribs can be connected to the backbone portions by one or more means (e.g., adhesive, solder, weld bead, mechanical connection, melted connected, compression connection, etc.). The backbone portion is illustrated in FIGS. 2 and 3 as having a generally rectangular shape; however, this is not required. The backbone portion is illustrated has extending the full longitudinal length of the medical device; however, this is not required. The thickness, width and shape of the backbone portion are non-limiting. The material used to form the backbone portion is non-limiting. The backbone portion can be a solid piece of material, can have one or more openings, be a mesh design, etc. As illustrated in FIGS. 2 and 3, the backbone portion is illustrated as a solid single piece of material.

Referring again to FIG. 3, a plurality of ribs 30 are connected to or formed on each side of the backbone portion. As illustrated in FIG. 3, four ribs are positioned on each side of the backbone portion. As can be appreciated, each side of the backbone portion can include more than four ribs (e.g., 5-20, etc.) or less than four ribs (e.g., 1-3). The ribs are illustrated as being spaced apart at equal distances; however, this is not required. The ribs are illustrated as being the same shape, width, thickness and length; however, this is not required. The length, width, thickness and shape of the rib portions are non-limiting. As illustrated in FIG. 3, the ribs are position diametrically opposite of one another; however, this is not required.

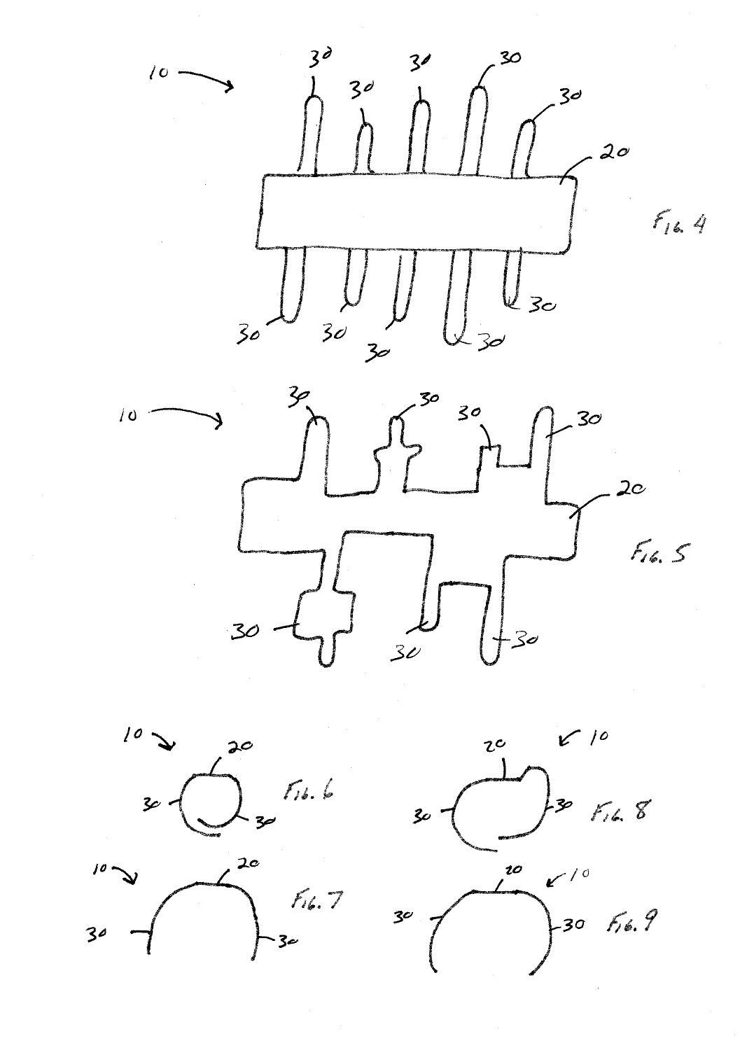

FIGS. 4 and 5 illustrate additional non-limiting configurations of the medical device of the present invention. In FIG. 4, the backbone portion again has a generally rectangular shape and extends the full longitudinal length of the medical device. The number of ribs 30 on each side of the backbone portion is the same and the ribs are position diametrically opposite of one another. The size of the ribs are illustrated as being different. In FIG. 5, the shape of the backbone position is not rectangular. The number of ribs on each side of the backbone portion is not the same. The shape, size and length of the ribs on each side of the backbone portion are different. Also, the ribs are not position diametrically opposite of one another. As can be appreciated, the medical device of the present invention can have other configurations.

Referring now to FIG. 1, the end portion of one or more of the ribs can include a curved or flared portion 32. The orientation of the curved portion is non-limiting. The curved portion, when used, is designed to inhibit or prevent the ends of the ribs from penetrating into the body passageway when the medical device is expanded. Generally, the curved portion, when used, forms less than a majority of the length of the rib; however, this is not required. The shape of the curved portion, when used, is non-limiting.

As illustrated in FIG. 1, the width of the backbone portion is generally selected such that when the medical device is in the expanded position, the width of the backbone portion constitutes less than a majority of the outer perimeter of the medical device; however, this is not required. As also illustrated in FIG. 1, when the medical device is in the expanded position, all or a majority of the end of the ribs are spaced from one another and do not overlap one another; however, this is not required.

FIGS. 6-9 illustrate various embodiments of the medical device of the present invention in the expanded and non-expanded position. FIGS. 6 and 8 illustrate the medical device in the non-expanded position. FIGS. 7 and 9 illustrate the medical device in the expanded position. The manner in which the medical device is folded or otherwise formed after the medical device has been formed (e.g., stamped, laser, cut, molded, soldered, welded, etc.) is non-limiting. As illustrated in FIGS. 6 and 8, the medical device is folded into different configurations. As illustrated in FIGS. 7 and 9, the medical device has been expanded (e.g., by a balloon, etc.) to cause the ends of the ribs to be spaced from one another and to be in a non-overlapped position.

Prior to the medical device being inserted into the body passageway, the ribs and/or the backbone portion are bent so that the ends of the ribs 30 overlap one another or are positioned adjacent to one another. The materials used to form the rib portions and/or the backbone portion is non-limiting (e.g., metal, polymer, etc.).

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween.

1-24. (canceled)

25. A medical device that can be expanded between an non-expanded and expanded position in a body passageway, said medical device designed to be inserted into a body passageway and to a treatment region in the body passageway while the medical device is in the non-expanded position, said medical device designed to be expandable while in the body passageway, said medical device including a backbone portion and a plurality of sets of ribs connected to said backbone portion, said backbone portion extending at least a majority of a longitudinal length of said medical device, each set of ribs includes a first and second rib, said first rib positioned on one side of said backbone portion, said second rib positioned on an opposite side of said backbone portion, an end of said first and second ribs are spaced from one another and do not overlap one another when the medical device is in the expanded position.

26. The medical device as defined in claim 25, wherein said first and second ribs are positioned diametrically apart from one another.

27. The medical device as defined in claim 25, wherein said first and second ribs are not positioned diametrically apart from one another.

28. The medical device as defined in claim 25, wherein said first and second ribs have the same length and shape.

29. The medical device as defined in claim 26, wherein said first and second ribs have the same length and shape.

30. The medical device as defined in claim 25, wherein said first and second ribs have a different length and/or shape.

31. The medical device as defined in claim 25, wherein said first and second ribs in a first set of ribs have a same length and shape as said first and second ribs in a second set of ribs.

32. The medical device as defined in claim 29, wherein said first and second ribs in a first set of ribs have a same length and shape as said first and second ribs in a second set of ribs.

33. The medical device as defined in claim 25, wherein said first and second ribs in a first set of ribs have a different length, shape, or combinations thereof from said first and second ribs in a second set of ribs.

34. The medical device as defined in claim 32, wherein said backbone extends the full longitudinal length of said medical device.

35. The medical device as defined in claim 34, wherein said backbone is formed of a single piece of material and has a generally rectangular shape.

37. The medical device as defined in claim 25, wherein said backbone has a width that consists of no more than about 75% of an outer circumference of said medical device in said expanded position.

38. The medical device as defined in claim 35, wherein said backbone has a width that consists of no more than about 75% of an outer circumference of said medical device in said expanded position.

39. The medical device as defined in claim 25, wherein said end of a plurality of said ribs are disconnected from one another and overlap a portion of an adjacently positioned rib when said medical device is in said non-expanded position.

40. The medical device as defined in claim 38, wherein said end of a plurality of said ribs are disconnected from one another and overlap a portion of an adjacently positioned rib when said medical device is in said non-expanded position.

41. The medical device as defined in claim 25, wherein an end of at least one of said ribs includes a curved portion that is designed to inhibit penetration of an end of said rib into said body passageway when said medical device is expanded into said expanded position.

42. The medical device as defined in claim 40, wherein an end of at least one of said ribs includes a curved portion that is designed to inhibit penetration of an end of said rib into said body passageway when said medical device is expanded into said expanded position.

43. The medical device as defined in claim 42, wherein each of said ribs has a length that consists of no more than about 20-40% of the outer circumference of the medical device in the fully expanded position.

44. The medical device as defined in claim 25, wherein adjacently positioned sets of ribs are spaced equal distances from another, said backbone including at least four sets of ribs.

45. The medical device as defined in claim 43, wherein adjacently positioned sets of ribs are spaced equal distances from another, said backbone including at least four sets of ribs.

46. The medical device as defined in claim 25, wherein said end of a plurality of said ribs are releasably connected to one another and overlap a portion of an adjacently positioned rib when said medical device is in said non-expanded position.

47. A method for treating an area in an interior of a body passageway comprising:
a. providing a medical device in a non-fully expanded position, said medical device designed to be inserted into a body passageway and to a treatment region in the body passageway while the medical device is in said non-fully expanded position, said medical device designed to be expandable while in the body passageway, said medical device including a backbone portion and a plurality of sets of ribs connected to said backbone portion, said backbone portion extending at least a majority of a longitudinal length of said medical device, each set of ribs includes a first and second rib, said first rib positioned on one side of said backbone portion, said second rib positioned on an opposite side of said backbone portion;
b. inserting said medical device into said body passageway while said medical device is in said non-fully expanded position; and,
c. expanding said medical device in said body passageway until an end of said first and second ribs are spaced from one another and do not overlap one another.
